(19) United States
(12) Patent Application Publication
(10) Pub. No.: US 2026/0298916 A1
(43) Pub. Date: Oct. 1, 2026

(54) OLIGOSACCHARIDE AND/OR POLYSACCHARIDE COATED IRON OXIDE NANORODS AND METHODS OF MAKING AND USE THEREOF

(71) Applicant: 5M BIOMED, LLC, Johns Creek, GA (US)

(72) Inventors: Yuancheng LI, Atlanta, GA (US); Hui MAO, Johns Creek, GA (US)

(21) Appl. No.: 19/481,185

(22) PCT Filed: Nov. 10, 2023
(86) PCT No.: PCT/US2023/079303

§ 371 Date: Nov. 4, 2025

Related U.S. Application Data
(60) Provisional application No. 63/500,396, filed on May 5, 2023.

(51) Int. Cl. G01N 33/543; A61K 49/18; B82Y 15/00; G01N 33/531

(52) U.S. Cl. CPC G01N 33/54333; A61K 49/1884; G01N 33/531; G01N 33/5434; A61K 2123/00; B82Y 15/00; G01N 2446/86

(57) ABSTRACT

Described are coated iron oxide nanorods (IONRs) containing an iron oxide core and a coating surrounding the core, and pharmaceutical compositions containing these coated IONRs. The iron oxide core of the coated IONRs has strong magnetic property, i.e., a magnetic flux density of at least 10 emu/g, induced using 1 T magnetizing field strength, at room temperature. The coating of the coated IONRs can be formed by a sugar in situ on the surface of the iron oxide corn. The coated IONRs are stable in an aqueous medium for at least 30 mins, at room temperature, while maintain the superior magnetic property of the core, achieving a separation efficiency of at least 80% within only 1 min of magnet time. Optionally, the coated IONRs contain one or more active agents embedded in the coating of the coated IONRs, for systemic or local delivery.

### CROSS-REFERENCE TO RELATED APPLICATIONS

This application claims the benefit of and priority to U.S. Provisional Application No. 63/500,396 filed May 5, 2023, the entire content of which is incorporated herein by reference for all purpose in its entirety.

### FIELD OF THE INVENTION

This invention is generally in the field of iron oxide nanorods coated with oligosaccharide and/or polysaccharide and methods of making and using thereof.

### BACKGROUND OF THE INVENTION

Iron oxide nanoparticles are widely used magnetic materials for developing molecular imaging probes, in vitro diagnostic agents, image-guided drug delivery, and hyperthermia treatments. In particular, magnetic iron oxide nanorods (IONRs) have gained considerable interest owning to their high relaxivities enhancing magnetic resonance imaging (MRI) contrast. In in vivo applications, anisotropic nanoparticles exhibit favorable pharmacokinetics, higher cell uptake, and improved tumor targeting and intratumoral retention time. In in vitro applications, IONRs with much stronger magnetism allow fast and efficient immunomagnetic separation. However, the use of IONRs in biomedical applications has been hampered by their instability in aqueous medium. The use of coating materials may improve the nanorods' aqueous stability. However, current coated IONRs are limited to those having relatively weak magnetic properties, because IONRs having strong magnetic properties (i.e., a magnetic moment ≥10 emu/g, induced using 1 T magnetizing field strength, at room temperature) tend to form clusters in the coating process, which makes coating individual IONR very challenging.

There remains a need for coated iron oxide nanorods that are stable in aqueous medium and have strong magnetic properties and methods for making such.

Therefore, it is the object of the present invention to provide coated iron oxide nanorods that are stable in aqueous medium and have strong magnetic properties.

It is another object of the present invention to provide methods for making the coated iron oxide nanorods.

It is another object of the present invention to provide methods for using the coated iron oxide nanorods.

### SUMMARY OF THE INVENTION

Disclosed are coated iron oxide nanorods (IONRs), pharmaceutical compositions containing these coated IONRs, and methods of making and using these coated IONRs. The disclosed coated IONRs contain an iron oxide core and a coating. The iron oxide core has a magnetic moment of at least 10 emu/g, induced using 1 T magnetizing field strength, at room temperature. The coated IONRs disclosed herein have excellent aqueous stability (i.e., stay suspended in an aqueous medium for at least 1 hour, at room temperature), while maintaining the superior magnetic property of the IONRs (i.e., a magnetic moment of at least 10 emu/g, induced using 1 T magnetizing field strength, at room temperature). This combined features lead to a separation efficiency of these coated IONRs of at least 80%, within 1 min of magnet time, and optionally at least 90%, within 2 min of magnetic time.

Typically, the iron oxide core of the coated IONRs contains Fe₃O₄ magnetite or a combination of Fe₃O₄ magnetite and FeO(OH) goethite. Generally, the iron oxide core has a rod shape, with a length in a range from about 20 nm to about 250 nm and a width in a range from about 2 nm to about 50 nm.

The coating of the coated IONRs can be formed using any suitable carbohydrate, such as one or more oligosaccharides or polysaccharides, or a combination thereof. In some forms, the coating of the coated IONRs is formed from one or more oligosaccharides. In some forms, the coating of the coated IONRs is formed from one or more polysaccharides. In some forms, the coating of the coated IONRs is formed from a combination of oligosaccharides and oligosaccharides. In some forms, the coating of the coated IONRs is formed from a combination of monosaccharide, oligosaccharides and oligosaccharides. Typically, the oligosaccharide and/or polysaccharide coating is formed in situ on the surface of the core through condensation reactions of simple sugar molecules, such as glucose.

The coated IONRs have hydrodynamic dimensions, which can be measured using methods known in the art, such as dynamic light scattering. Generally, the coated IONRs have a hydrodynamic length in a range from about 50 nm to about 300 nm, from about 100 nm to about 300 nm, from about 50 nm to about 200 nm, from about 50 nm to about 150 nm, from about 150 nm to about 300 nm, from about 200 nm to about 300 nm, or from about 220 nm to about 300 nm, and a hydrodynamic width in a range from about 20 nm to about 150 nm, from about 20 nm to about 120 nm, from about 20 to about 100 nm, or from about 50 nm to about 150 nm. Further, in some forms, the coated IONRs have a zeta potential in a range from about −10 mV to about −100 mV measured in double distilled water.

Kits and devices containing the coated IONR(s) are disclosed. The kit includes a device and the coated IONR(s). The device in the kit contains one or more defined region(s) or well(s), which are configured to contain a sample for in vitro applications, such as cell separation or target cell detection. Typically, in the kit, the coated IONR(s) are stored separately from the device prior to use. In some forms, the user is provided with just a device. The device contains one or more defined region(s) or well(s) and the coated IONR(s), wherein each defined region or well contains the IONRs.

Methods of producing the disclosed coated iron oxide nanorods (IONRs) are also described. Generally, the method includes (i) mechanically mixing (such as by inverting, shaking, or stirring) a mixture containing IONRs, a coating precursor, and a solvent for a time period in a range from about 1 hour to about 10 hours, from about 1 hour to about 8 hours, from about 1 hour to about 5 hours, from about 2 hours to about 10 hours, from about 2 hours to about 8 hours, or from about 2 hours to about 5 hours, at a temperature in a range from about 60° C. to about 150° C., from about 80° C. to about 150° C., from about 100° C. to about 150° C., such as about 120° C., to form a product containing the coated IONRs; and (ii) during step (i), periodically sonicating the mixture for at least 1 min, at least 2 mins, at least 5 mins, at least 10 mins, at least 20 mins, at least 30 mins, or in a range from 1 min to 1 hour, from 1 min to 30 mins, from 1 min to 20 mins, from 1 min to 10 mins, or from 1 min to 5 mins, at room temperature. Step (ii) can be performed at a regular time interval or irregular time interval during step (i) mechanical stirring the mixture. For example, step (ii) is performed regularly every 30 mins, every hour, or every 2 hours during step (i), or step (ii) is performed irregularly with a time interval of 30 mins, 1 hour, and/or 2 hours.

Optionally, the method further includes prior to step (i), one or more of the following steps: (a) sonicating a stock solution containing IONRs and the solvent for a period in a range from about 1 min to about 1 hour, from about 1 min to about 50 mins, from about 1 min to about 40 mins, from about 1 min to about 30 mins, from about 1 min to about 20 mins, or from about 1 min to about 10 mins, prior to step (i); (b) dissolving a coating precursor in a solvent to form a coating precursor solution at a suitable temperature, such as a temperature ranging from about 60° C. to about 100° C., such as about 90° C., prior to step (i); (c) adding the sonicated stock solution into the coating precursor solution, such as by using a syringe pump at a suitable speed (such as a speed ranging from about 0.1 ml/min to 1 ml/min, such as about 0.35 ml/min), to form the mixture, prior to step (i); (d) heating the mixture to the temperature in step (i) (e.g., the mixture is heated such that the temperature is gradually raised to the temperature in step (i)); and optionally, subsequently, (e) collecting coated IONRs, such as by washing the product using a wash solvent (such as ethanol) and subjecting the washed product to ultracentrifugation, where the washing and ultracentrifugation are repeated one or more times, such as one, two, three, four, or five times.

The disclosed IONRs can be used in applications such as in purifying target cells, detecting target cells, in vivo or in vitro, delivering active agents, and imaging (as contrast agents).

### BRIEF DESCRIPTION OF THE DRAWINGS

FIGS. 1A-1C are images of uncoated IONRs synthesized following protocol 1 (FIG. 1A) and protocol 2 (FIG. 1B and FIG. 1C), measured using transmission electron microscopy (TEM). FIG. 1D and FIG. 1E are graphs showing the XRD (X-Ray Power Diffraction result of uncoated IONRs synthesized following protocol 1 (FIG. 1D) and protocol 2 (FIG. 1E).

FIGS. 2A and 2B are TEM images of oligosaccharide/polysaccharide coated IONRs (where the IONRs were synthesized following protocol 2). FIG. 2C is a graph showing the dynamic light scattering measurement results of the oligosaccharide/polysaccharide coated IONRs. FIG. 2D is a graph showing the self-precipitation profiles of the oligosaccharide/polysaccharide coated IONRs and commercially available magnetic materials (i.e., from Miltenyi Biotech, Dynabead®, and from StemCell Technology). FIG. 2E is a graph showing the separation efficiency by magnet of the oligosaccharide/polysaccharide coated IONRs and commercially available magnetic materials (i.e., from Miltenyi Biotech, Dynabead®, and StemCell Technology). FIG. 2F is a graph showing the magnetic moment of the oligosaccharide/polysaccharide coated IONRs.

### DETAILED DESCRIPTION OF THE INVENTION

### I. Compositions

Oligosaccharide/polysaccharide coated iron oxide nanorods ((also referred to herein as “coated IONRs”) and compositions containing these coated IONRs for in vivo and in vitro applications are described herein. The coated IONRs described herein generally demonstrate stability in an aqueous medium (i.e., stay suspended in the aqueous medium for at least 1 hour, at room temperature) and a strong magnetic property (i.e., a magnetic moment of at least 10 emu/g, induced using 1 T magnetizing field strength, at room temperature). The aqueous stability and superior magnetic property of the disclosed coated IONRs make them suitable for use in a wide range of in vivo and in vitro biomedical applications, such as for use in purifying and detecting one or more target biological substances (such as proteins, peptides, DNAs, RNAs, cells, extracellular vesicles, cytokines, etc.), in vivo or in vitro, delivering active agents, and imaging as contrast agents.

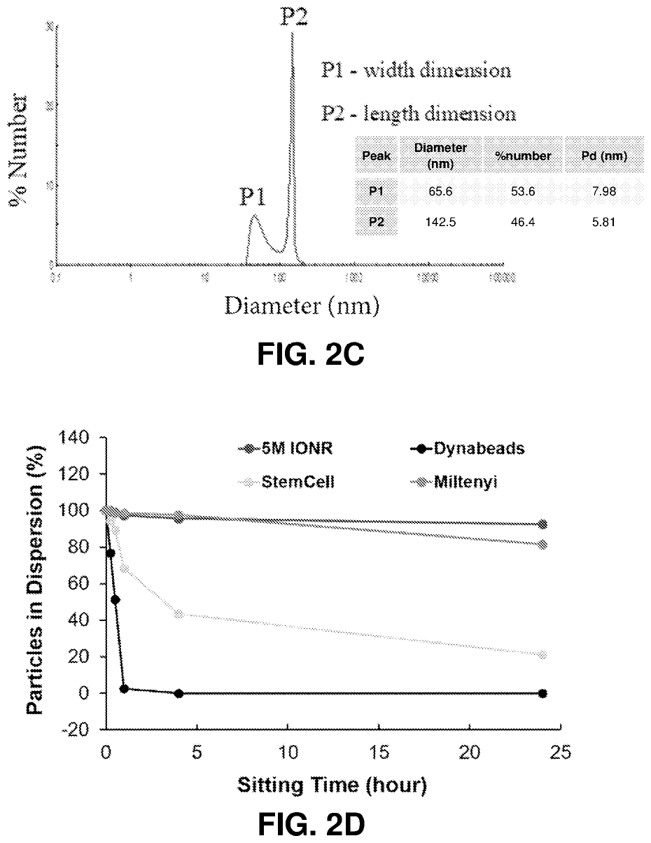

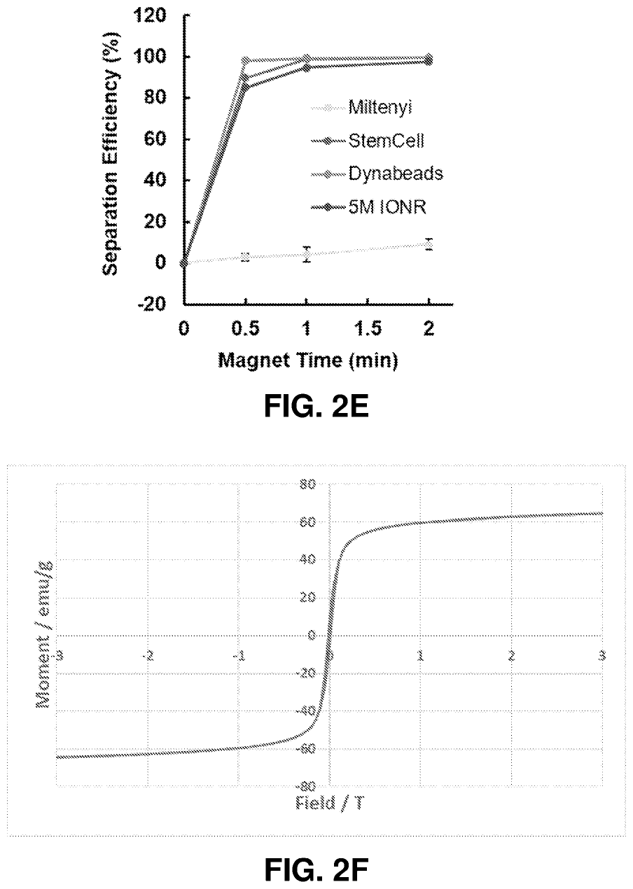

By having the combined properties of aqueous stability and superior magnetic property, the IONRs disclosed herein outperform commercially available iron oxide materials, such as Dynabeads®, the magnetic beads available from StemCell Technology, and Miltenyi Biotec, which only present one of these two properties of the coated IONRs. Coating iron oxide particles with strong magnetic properties is a major limitation in the applications of such materials, because they tend to aggregate and form clusters in the coating process, which results in insufficient coating of these materials and thus poor aqueous stability (for example, Dynabeads® and magnetic beads available from StemCell Technology). To achieve sufficient coating of iron oxide particles, currently available materials use iron oxide particles with relatively weak magnetic property, leading to coated materials with low separation efficiency (for example, magnetic beads available from/supplied by Miltenyi Biotec). These issues are addressed by the coated IONRs disclosed herein. For example, as shown in FIGS. 2D-2F and described in the Examples below, the coated IONRs are stable in an aqueous medium for a time period that is longer than Dynabeads® and the magnetic beads from StemCell Technology under the same measurement conditions, and has a separation efficiency that is higher than the magnetic beads from Miltenyi Biotech under the same measurement conditions. The term “same measurement conditions” refers to measurement in the same aqueous medium, pre-dispersed in the aqueous medium using the same sonication method and same sonication time, and measured UV absorbance at the same temperature, pressure, time point, etc.

When the coated IONRs are tested for their separation performance in vitro, such as by pulling out the coated IONRs dispersed in an aqueous medium using a magnet, the separation efficiency can reach an appreciable level, such as at least 80%, at least 85%, at least 90%, or at least 95%, within 1 min of magnet time. The term “magnet time” refers to the time period of which the coated IONRs are exposed to a magnetic field. The term “separation efficiency” refers to the amount of the dispersed coated IONRs that is pulled out from an aqueous medium by a magnet force using a magnet, relative to the amount of the coated IONRs dispersed in the aqueous medium prior to applying the magnet force, as determined using UV-vis absorbance at room temperature.

### A. Coated Iron Oxide Nanorods

The oligosaccharide/polysaccharide coated IONRs disclosed herein contain an iron oxide core and a oligosaccharide/polysaccharide coating. The coating surrounds the iron oxide core. Without being bound by any theories, the oligosaccharide/polysaccharide coating may be chemically attached or physically absorbed to the surface of the iron oxide core, or a combination thereof. The disclosed IONRs not only have excellent aqueous stability (i.e., stay suspended in the aqueous medium for at least 1 hour, at room temperature), but also show superior magnetic property (i.e., a magnetic moment of at least 10 emu/g, induced using 1 T magnetizing field strength, at room temperature) and consequently, can achieve a separation efficiency of at least 80% within 1 min of magnet time.

### 1. Iron Oxide Core

The iron oxide core of the coated IONR has the shape of a rod (also referred to herein as “iron oxide nanorod” or “uncoated iron oxide nanorod”) and provides strong magnetic property (i.e., a magnetic moment of at least 10 emu/g, induced using 1 T magnetizing field strength, at room temperature). For example, the iron oxide core of the coated IONRs has a magnetic moment of at least 20 emu/g, at least 50 emu/g, or at least 80 emu/g, as determined using hysteresis loops, induced using 1 T magnetizing field strength, at room temperature. It is understood that other magnetic materials may be used to replace the iron oxide in the core and then coated with a coating material to form coated nanorods. Examples of other magnetic materials (in place of iron oxide) suitable for forming the core include, but are not limited to, cobalt oxide, nickel oxide, titanium oxide, and ferrites (e.g., ZnFe₂O₄, SrFe₁₂O₁₉, BaFe₁₂O₉, and CoFe₂O₄).

The iron oxide core can have any suitable dimensions, depending on the specific applications. Generally, the iron oxide core has a length from about 20 nm to about 250 nm and optionally a width from about 2 nm to about 50 nm. For example, the iron oxide core has a length from about 20 nm to <200 nm, from about 50 nm to about 150 nm, from about 150 nm to about 250 nm, from about 150 nm to about 200 nm, or from about 180 nm to about 250 nm. In some forms, the dimensions of the iron oxide core were measured using a plurality of the core and the dimensions thus refer to the average dimensions. For example, the iron oxide core has an average length from about 20 nm to <200 nm, from about 50 nm to about 150 nm, from about 150 nm to about 250 nm, from about 150 nm to about 200 nm, or from about 180 nm to about 250 nm, and optionally an average width from about 2 nm to about 50 nm. The dimensions of a coated IONR can be measured using methods known in the art, such as transmission electron microscopy (“TEM”) and dynamic light scattering (“DLS”). For example, for measuring the physical dimensions of the coated IONRs, TEM is typically used, while for measuring the hydrodynamic dimensions of the coated IONRs, DLS is typically used.

The iron oxide in the iron oxide core can be in a variety of forms, such as magnetite (Fe₃O₄), maghemite (Fe₂O₃), or goethite (FeO(OH), or a combination thereof. In some forms, the iron oxide in the core is magnetite (Fe₃O₄). In some other forms, the iron oxide in the core is a combination of magnetite (Fe₃O₄) and goethite (FeO(OH)). When the iron oxide in the core has more than one form, the content of each form can be in any suitable amount. For example, when the iron oxide in the core is magnetite (Fe₃O₄), the content of the magnetite can be from about 95% to 100%. For example, when the iron oxide in the core is magnetite (Fe₃O₄) and goethite (FeO(OH)), the content of the magnetite can be from about 40% to about 80%, such as about 50%, and the remaining balance is goethite (i.e., in a range from about 60% to about 20%, such as bout 50%), for example, about 53% magnetite and about 47% goethite.

### 2. Coating

The coating of the coated IONRs is an oligosaccharide or a polysaccharide, or a combination thereof. The coating surrounds the iron oxide core and may be chemically attached or physically absorbed to the surface of the iron oxide core, or a combination thereof. An “oligosaccharide” refers to a carbohydrate chain composed of three to ten monosaccharides. An “polysaccharide” refers to a carbohydrate chain composed of more than ten monosaccharides.

Typically, the oligosaccharide/polysaccharide coating is formed in situ on the surface of the iron oxide core via a coating reaction of a sugar, such as a monosaccharide, a disaccharide, or a combination thereof. Examples of sugars suitable for forming the oligosaccharide/polysaccharide coating of the coated IONRs include, but are not limited to, glucose, fructose, galactose, ribose, and xylose, and combinations thereof. When the oligosaccharide/polysaccharide coating is formed by two or more sugars, each sugar can be present in any suitable ratio.

The chain of the oligosaccharide can contain the same sugar (e.g., the same monosaccharide or the same disaccharide) or two or more different sugars (e.g., two or more different monosaccharides, two or more different disaccharides, or a monosaccharide and a disaccharide where the disaccharide is formed from a different monosaccharide). For example, the oligosaccharide coating of the coated IONRs contains three to ten of the same monosaccharide, such as glucose, fructose, galactose, ribose, or xylose. For example, the oligosaccharide forming the coating of the coated IONRs contains three to ten monosaccharides, where at least one monosaccharide is different from the other monosaccharides in the chain.

Similarly, the chain of the polysaccharide can contain the same sugar (e.g., the same monosaccharide or the same disaccharide) or two or more different sugars (e.g., two or more different monosaccharides, two or more different disaccharides, or a monosaccharide and a disaccharide where the disaccharide is formed from a different monosaccharide). For example, the polysaccharide coating of the coated IONRs contains ten or more of the same monosaccharide, such as glucose, fructose, galactose, ribose, or xylose. For example, the polysaccharide forming the coating of the coated IONRs contains ten or more monosaccharides, where at least one monosaccharide is different from the other monosaccharides in the chain.

In some forms, the coating precursor, such as the hydroxyl groups of the sugar, is modified such that it contains one or more functional moieties, such as amino, carboxyl, thiol, and/or maleimide. When forming a oligosaccharide/polysaccharide coating surrounding the iron oxide core, these functional moiety(ies) are incorporated on the surface of the iron oxide core. Additionally or alternatively, the sugar forming the coating is not modified and the hydroxyl group(s) of the oligosaccharide/polysaccharide coating can be converted into other functional groups, such as amino, carboxyl, thiol, and/or maleimide groups.

The functional groups incorporated on the surface of the iron oxide core can be further modified, for example, to attach a targeting moiety. The targeting moiety can be any suitable molecules (such as NTA-Ni) or entity capable of binding a biological substance, such as proteins (e.g., antibodies), peptide, extracellular vesicles, or a surface marker that is expressed by a specific cell population, e.g., lymphocytes, monocytes, stem cells, or tumor cells. Examples of targeting moieties include, but are not limited to, an antibody, an antibody fragment, an antibody mimetic, an affibody, a nucleic acid, an oligonucleotide, an aptamer, a peptide, a steroid, and a tyrosine kinase inhibitor. More specific examples of suitable targeting moieties are described in U.S. Pat. No. 10,393,736, the disclosure of which is incorporated herein by reference in its entirety.

a. Active Agents

Optionally, the coated IONRs contain one or more active agents embedded in the coating. The active agent(s) embedded in the coating of the coated IONRs may be hydrophilic or hydrophobic, depending on the components and the structure of the coating. For example, in some forms, the coating is formed by glucose and the active agents contained in the coated IONRs are typically hydrophilic.

The active agent or each active agent of two or more active agents within the coated IONRs can be a therapeutic agent, a diagnostic agent, or a prophylactic agent. Active agents with a wide range of molecular weight can be loaded in the coated IONRs, for example, between 100 Da and 10,000 kDa. Examples of active agents that can be loaded in the coated IONRs for delivery include, but are not limited to, small molecules, proteins, polypeptides, peptides, carbohydrates, nucleic acids, glycoproteins, lipids, and antibodies/antigens, and combinations thereof. “Small molecule” generally refers to an organic molecule that is less than about 900 Da. Typically, small molecules are non-polymeric and/or non-oligomeric.

The weight percentage of the active agent or the total weight percentage of the two or more active agents in the coated IONRs, when present, depends on the hydrophilicity/hydrophobicity and the molecular weights of the active agents. Optionally, the weight percentage of the active agent or the total weight percentage of the two or more active agents in the coated IONRs is in a range from 0.01% to 80%, from 0.01% to 60%, from 0.01% to 50%, from 0.01% to 40%, from 0.01% to 25%, from 0.01% to 10%, from 0.01% to 5%, from 0.1% to 50%, from 0.1% to 25%, from 0.1% to 10%, from 0.1% to 5%, from 0.05% to 10%, from 0.05% to 5%, from 0.01% to 2%, from 0.05% to 2%, from 0.1% to 2%, from 0.01% to 1%, from 0.05% to 1%, or from 0.1% to 1%. The term “weight percentage of the active agent” refers to the weight of the active agent relative to the sum of the weights of the active agent, the coating, and the core. The term “total weight percentage of the two or more active agents” refers to the sum of the weights of the active agents relative to the sum of the weights of the active agents, the coating, and the core.

The weight percentage of the active agent or total weight percentage of the two or more active agents can be varied based on the specific agent(s) being delivered. For example, for large biomolecules, such as proteins and nucleic acids, typical weight percentages of the large biomolecule in the coated IONRs are from 0 0.01% to 20%, from 0.01% to 5%, from 0.01% to 2.5%, or from 0.01% to 1%.

Examples of active agents and their alternative forms such as alternative salt forms, free acid forms, free base forms, and hydrates that can be loaded in the coating of the coated IONRs for delivery include, but are not limited to, anticancer agents (such as those described in U.S. Pat. No. 10,393,736, the disclosure of which is incorporated herein by reference in its entirety); analgesics/antipyretics (such as aspirin, acetaminophen, ibuprofen, etc.); antibiotics; antidepressants; antidiabetics; antifungal agents; antihypertensive agents; anti-inflammatories; antianxiety agents; immunosuppressive agents; antimigraine agents; sedatives/hypnotics; antianginal agents; antipsychotic agents; antimanic agents; antiarrhythmics; antiarthritic agents; antigout agents; anticoagulants; thrombolytic agents; antifibrinolytic agents; hemorheologic agents; antiplatelet agents; anticonvulsants; antiparkinson agents; antihistamines/antipruritics; agents useful for calcium regulation; antibacterial agents; antiviral agents; antimicrobials; anti-infectives; bronchodilators; steroidal compounds, hormones and hormone analogues; hypoglycemic agents; hypolipidemic agents; peptides, proteins, and nucleic acids; agents useful for erythropoiesis; antiulcer/anti-reflux agents; antinauseants/antiemetics; and vitamins. Any of these active agents or a combination thereof can be loaded in the coated IONRs for delivery.

In some forms, the active agent(s) loaded in the coating of the coated IONRs for delivery is/are anticancer agent(s), such as temozolomide, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin and/or lenalidomide or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); and methotrexate, vincristine, doxorubicin, cisplatin (MVAC); and combinations thereof.

### 3. Properties

The performance of the coated IONRs can be evaluated by the properties of these materials, such as hydrodynamic dimensions, zeta potential, magnetic properties, dispersion in aqueous medium, and separation efficiency.

a. Hydrodynamic Dimensions and Zeta Potential

The hydrodynamic dimensions of the coated IONRs reflects the hypothetical dimensions of an IONR moving in the solution. The coated IONRs typically have a hydrodynamic length and a hydrodynamic width, and can be measured in an aqueous medium using dynamic light scattering. Generally, the coated IONRs have a hydrodynamic length in a range from about 50 nm to about 300 nm, from about 100 nm to about 300 nm, from about 50 nm to about 200 nm, from about 50 nm to about 150 nm, from about 150 nm to about 300 nm, from about 200 nm to about 300 nm, or from about 220 nm to about 300 nm, and a hydrodynamic width in a range from about 20 nm to about 150 nm, from about 20 nm to about 120 nm, from about 20 to about 100 nm, or from about 50 nm to about 150 nm.

In some forms, the hydrodynamic dimensions of the coated IONR are measured using a plurality of coated IONRs and thus reflect the average of the hydrodynamic dimensions of the plurality of coated IONRs. In these forms, the coated INOR can have an average hydrodynamic length in a range from about 50 nm to about 300 nm, from about 100 nm to about 300 nm, from about 50 nm to about 200 nm, from about 50 nm to about 150 nm, from about 150 nm to about 300 nm, from about 200 nm to about 300 nm, or from about 220 nm to about 300 nm, and an average hydrodynamic width in a range from about 20 nm to about 150 nm, from about 20 nm to about 120 nm, from about 20 to about 100 nm, or from about 50 nm to about 150 nm.

The physical dimensions, i.e., the length and width, of the coated IONRs can be measured using the TEM, which measures the actual dimensions of the coated IONRs in solid state. Generally, the coated IONRs have an aspect ratio (length/width) ranging from 2 to 20, from 5 to 20, from 2 to 15, from 2 to 10, from 5 to 15, from 5 to 10, or from 6 to 9.

The material forming the coating of the coated IONRs can be positively charged, negatively charged, or have a near-neutral charge, depending on the types and numbers of surface functional groups. In some forms, the surface charge of the coated IONRs is near neutral (i.e., from −10 mV to +10 mV). In some forms, the material forming the coating of the coated IONRs is negatively charged or contain(s) one or more moieties (i.e. one or more functional groups, for example, amino groups) that impart a negative charge to the coating material in water. In these forms, when the iron oxide core is coated, the coating surrounding the core can provide a negative zeta potential for the coated IONR, measured in double distilled water at room temperature using a Zetasizer or similar instrument such as Zetaview. For example, the coated IONRs can have a zeta potential in a range from about −10 mV to −100 mV, in DI water at room temperature. The term “room temperature” refers to a temperature in a range from 20° C. to 25° C., such as about 25° C., at 1 atm.

An uncoated IONRs (i.e., iron oxide core) cannot be dispersed in aqueous medium sufficiently long for measurements of hydrodynamic dimensions and zeta potential. Thus, a successful measurement of hydrodynamic dimensions and/or zeta potential of the material in an aqueous medium, such as double distilled water, is a way of demonstrating that a coating formed around the iron oxide core.

b. Magnetic Properties

The magnetic properties of the coated IONRs can be determined using hysteresis curve, measured by a superconducting quantum interference device (SQUID). A hysteresis curve shows the magnetic moment (6, emu/g) of the material being measured at different applied magnetizing field strength. A magnetic moment ≥10 emu/g, induced using 1 T magnetizing field strength, at room temperature, is considered as having strong magnetic property. The coated IONRs typically have a magnetic moment of at least 10 emu/g, at least 20 emu/g, at least 50 emu/g, at least 80 emu/g, induced using 1 T magnetizing field strength, at room temperature. Such a magnetic moment of the disclosed coated IONRs is significantly stronger than those disclosed in previous studies, see, e.g., U.S. Pat. No. 9,125,941. For example, the oligosaccharide/polysaccharide coated IONRs have magnetic moment of at least 50 emu/g or at least 75 emu/g, induced using 1T magnetizing field strength, at room temperature.

c. Aqueous Stability

Self-precipitation of IONRs in aqueous medium is a main issue for their use in biomedical application. The coated IONRs disclosed herein have excellent aqueous stability. For example, the coated IONRs can stay suspended in the aqueous medium for at least 1 hour, at least 2 hours, at least 4 hours, at least 12 hours, at least 24 hours, at least 48 hours, at least 72 hours, at least 5 days, or at least 7 days, at room temperature, as determined using a UV-vis spectrometer. Compared to uncoated IONRs, the coated IONRs can stay suspended in the aqueous medium for a time period that is at least 50-time longer, at least 100-time longer, at least 200-time longer, or at least 1000-time longer, at room temperature, under the same measurement conditions.

In some forms, the aqueous stability of the coated IONRs is measured by dispersing a plurality of the coated IONRs in an aqueous medium and measuring the UV-vis change at different time point to determine the amount percentage of coated IONRs that remain suspended in the aqueous medium, relative to the amount of coated IONRs at time zero. Time zero is the UV-vis measurement made immediately (within 1 min) after dispersing the coated IONRs in the aqueous medium. For example, at least 90%, at least 95%, or at least 98% of the coated IONRs remain suspended in the aqueous medium at 4 hours, at room temperature, as determined using a UV-vis spectrometer. For example, at least 75%, at least 80%, or at least 85% of the coated IONRs that remain suspended in the aqueous medium at 48 hours, for 3 days, or for 7 days, at room temperature, as determined using a UV-vis spectrometer. Compared to uncoated IONRs, the amount/percentage of coated IONRs that remain suspended in the aqueous medium is at least 90% more, at least 95% more, or 100% more, at 4 hours, at room temperature, under the same measurement conditions; and/or at least 75%, at least 80%, or at least 85% more, at 48 hours, for 3 days, or for 7 days, at room temperature, under the same measurement conditions.

In some forms, the aqueous stability of the coated IONRs disclosed herein is superior to that of the commercially available magnetic beads, such as Dynabead® (Lot #: 00747022), the magnetic beads of supplied by StemCell Technology (Lot #: 1000096771), and/or Miltenyi Biotec (Lot #: 5221009844). For example, the coated IONRs can stay suspended in the aqueous medium for a period that is at least 50-time longer, 100-time longer, or 200-time longer, compared to Dynabead® (Lot #: 00747022), magnetic beads supplied by StemCell Technology (Lot #: 1000096771), and/or Miltenyi Biotec (Lot #: 5221009844), at room temperature, under the same measurement conditions. For example, the amount/percentage of coated IONRs that remain suspended in the aqueous medium is at least 40%, at least 60%, or at least 90% more than the amount/percentage of Dynabead® (Lot #: 00747022) and/or the magnetic beads supplied by StemCell Technology (Lot #: 1000096771), at room temperature, under the same measurement conditions; and/or at least 5%, at least 10%, at least 15%, or at least 20% more than the amount/percentage of the magnetic beads supplied by Miltenyi Biotec (Lot #: 5221009844), at room temperature, under the same measurement conditions.

An exemplary method of determining whether particles are suspended in an aqueous medium using UV-vis spectrometer is described below. For example, a solution of coated IONRs is vortexed and then set aside undisturbed to allow slow precipitation of coated IONRs out of suspension, if any. At different time points during setting, ranging from 1 min to 7 days, aliquots of the bulk solution are pipetted onto a 96 well plate, which is measured at 400 nm to obtain the absorbance values. The stability of the IONRs is calculated by comparing the absorbance at each time point vs that of the bulk solution taken after vortexing.

d. Separation Efficiency

In addition to the excellent aqueous stability described above, the disclosed coated IONRs also show superior in vitro separation efficiency. The separation efficiency can be measured by pulling out coated IONRs dispersed in an aqueous medium using a magnet, and the value is determined using the percentage of a plurality of dispersed coated IONRs that was pulled out from the aqueous medium within a short period of time, such as within 1 min of magnet time. Typically, the separation efficiency of the coated IONRs is at least 80%, at least 85%, at least 90%, or at least 95%, within 1 min of magnet time.

In some forms, the separation efficiency of the coated IONRs disclosed herein is higher than that of the commercially available magnetic beads, such as the magnetic beads of Miltenyi Biotec (Lot #: 5221009844). For example, the coated IONRs have a separation efficiency that is at least 10-time higher, 15-time longer, or 20-time higher, compared to the magnetic beads of Miltenyi Biotec (Lot #: 5221009844), under the same measurement conditions.

### B. Pharmaceutical Compositions Containing Coated IONRs

Pharmaceutical compositions that contain a plurality of the coated IONRs described herein in a form suitable for in vitro or in vivo applications (such as administration to a mammal), are disclosed. The pharmaceutical composition may include one or more pharmaceutically acceptable carriers and/or one or more pharmaceutically acceptable excipients. For example, the pharmaceutical composition may be in the form of a liquid, such as a solution or a suspension, and contain a plurality of the disclosed coated IONRs in an aqueous medium and, optionally, one or more suitable excipients for the liquid composition. Optionally, the pharmaceutical composition is in a solid form, and contains a plurality of the disclosed coated IONRs and one or more suitable excipients for a solid composition.

### 1. Carriers and Excipients

Suitable pharmaceutically acceptable carriers and excipients are generally recognized as safe (GRAS), and may be administered to an individual without causing undesirable biological side effects or unwanted interactions.

Representative carriers and excipients include solvents (including buffers), diluents, pH modifying agents, preservatives, antioxidants, suspending agents, wetting agents, viscosity modifiers, tonicity agents, and stabilizing agents, and a combination thereof.

Excipients can be added to a liquid or solid pharmaceutical composition (for in vivo or in vitro applications) to assist in sterility, stability (e.g. shelf-life), integration, and to adjust and/or maintain pH or isotonicity of the coated IONRs in the pharmaceutical composition, such as diluents, pH modifying agents, preservatives, antioxidants, suspending agents, wetting agents, viscosity modifiers, tonicity agents, and stabilizing agents, and a combination thereof.

Coated IONRs for administering active agents to a subject in need thereof, such as a mammal, can be dissolved or suspended in a suitable carrier to form a liquid pharmaceutical formulation, such as sterile saline, phosphate buffered saline (PBS), balanced salt solution (BSS), viscous gel, or other pharmaceutically acceptable carriers for administration. The pharmaceutical formulation may also be a sterile solution, suspension, or emulsion in a nontoxic, parenterally acceptable diluent or solvent.

### 2. Forms for Administration

A plurality of the disclosed coated IONRs can be formulated into a pharmaceutical formulation, in a liquid form or a solid form, as a liquid formulation or a solid formulation for oral administration or parenteral administration (e.g., intramuscular administration, intravenous administration, intraperitoneal administration, and subcutaneous administration) to a subject.

a. Oral Formulations

The pharmaceutical formulation containing a plurality of the disclosed coated IONRs may be provided in a form suitable for oral administration to a subject, such as a mammal (i.e., an oral composition). Oral administration may involve swallowing, so that coated IONRs, optionally encapsulating active agent(s) in the coating, enter the gastrointestinal tract, or buccal or sublingual administration may be employed by which the coated IONRs encapsulating active agent(s) enter the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays, and liquid formulations.

Liquid formulations for oral administration include suspensions, solutions, syrups, and elixirs. Such oral formulations may be employed as fillers in soft or hard capsules and can contain one or more suitable carriers and/or excipients, for example, water, ethanol, polyethylene glycol, propylene glycol, chitosan polymers and chitosan derivatives (e.g. N-trimethylene chloride chitosan, chitosan esters, chitosan modified with hydrophilic groups, such as amino groups, carboxyl groups, sulfate groups, etc.), methylcellulose, a suitable oil, one or more emulsifying agents, and/or suspending agents. Liquid formulations for oral administration may also be prepared by the reconstitution of a solid, for example, from a sachet.

Optionally, the coated IONRs are included in a fast-dissolving and/or fast-disintegrating dosage form.

For tablet or capsule dosage forms, in addition to the coated IONRs described herein, tablets generally contain disintegrants, binders, diluents, surface active agents, lubricants, glidants, antioxidants, colourants, flavouring agents, preservatives, or taste masking agents, or a combination thereof.

Examples of suitable disintegrants for forming a table or capsule dosage form containing the coated IONRs include, but are not limited to, sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant can have a concentration in a range from about 1 wt % to about 25 wt %, from about 5 wt % to about 20 wt % of the tablet or capsule dosage form containing the coated IONRs.

Binders are generally used to impart cohesive qualities to a tablet formulation containing the coated IONRs. Suitable binders for forming a tablet or capsule formulation containing the coated IONRs include, but are not limited to, microcrystalline cellulose, gelatin, sugars, polyethylene glycol, natural and synthetic gums, polyvinylpyrrolidone, pregelatinised starch, chitosan polymers and chitosan derivatives (e.g. N-trimethylene chloride chitosan, chitosan esters, chitosan modified with hydrophilic groups, such as amino groups, carboxyl groups, sulfate groups, etc.), hydroxypropyl cellulose, and hydroxypropyl methylcellulose.

Suitable diluents for forming a table or capsule formulation containing the coated IONRs include, but are not limited to, lactose (as, for example, the monohydrate, spray-dried monohydrate or anhydrous form), chitosan polymers and chitosan derivatives (e.g. N-trimethylene chloride chitosan, chitosan esters, chitosan modified with hydrophilic groups, such as amino groups, carboxyl groups, sulfate groups, etc.), N-sulfonated derivatives of chitosan, quaternarized derivatives of chitosan, carbosyalkylated chitosan, microcrystalline chitosan, mannitol, xylitol, dextrose, sucrose, sorbitol, microcrystalline cellulose, starch and dibasic calcium phosphate dihydrate.

Tablet or capsule formulation containing the coated IONRs may also contain surface active agents, such as sodium lauryl sulfate and polysorbate 80, and glidants such as silicon dioxide and talc, in the coating. When present, surface active agents can have a concentration in a range from about 0.2 wt % to 5 wt % of the tablet or capsule formulation.

Tablet or capsule formulations containing the coated IONRs also generally contain lubricants, such as magnesium stearate, calcium stearate, zinc stearate, sodium stearyl fumarate, and mixtures of magnesium stearate with sodium lauryl sulphate. Lubricants can have a concentration in a range from about 0.25 wt % to 10 wt %, from about 0.5 wt % to about 3 wt % of the tablet or capsule formulation.

Other possible excipients included in a tablet or capsule formulation containing the coated IONRs include glidants (e.g. Talc or colloidal anhydrous silica at about 0.1 wt % to about 3 wt % of the table or capsule formulation), antioxidants, colourants, flavouring agents, preservatives and taste-masking agents. When present, glidants can have a concentration in a range from about 0.2 wt % to 1 wt % of the tablet or capsule formulation.

An exemplary tablet formulation contains up to about 80 wt % of the coated IONRs described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet or capsule blends, including the coated IONRs and one or more suitable excipients, may be compressed directly or by roller to form tablets. Tablet or capsule blends or portions of the blends may alternatively be wet-, dry-, or melt-granulated, melt congealed, or extruded before tableting. The final table or capsule formulation may contain one or more layers and may be coated or uncoated; it may even be encapsulated in a particle, such as a polymeric particle or a liposomal particle.

Solid formulations containing the coated IONRs for oral administration may be formulated to be immediate and/or modified release. Modified release formulations include delayed, sustained, pulsed, controlled, targeted and programmed release formulations.

b. Parenteral Formulations

Optionally, the pharmaceutical formulation containing a plurality of the disclosed coated IONRs is in a form suitable for administration directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

For example, the pharmaceutical formulation containing a plurality of the coated IONRs is in a form suitable for intramuscular administration, intravenous administration, intraperitoneal administration, or subcutaneous administration, or a combination thereof.

Parenteral formulations containing the coated IONRs described herein are typically aqueous solutions which can contain excipients such as salts, carbohydrates and buffering agents (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4), but, for some applications, they may be more suitably formulated as a sterile aqueous solution or as a dried form to be used in conjunction with a suitable vehicle such as sterile, pyrogen-free water.

The liquid formulations containing the coated IONRs for parenteral administration may be a solution, a suspension, or an emulsion.

The liquid pharmaceutically acceptable carrier forming the parenteral formulation containing the coated IONRs can include one or more physiologically compatible buffers, such as a phosphate buffers. One skilled in the art can readily determine a suitable saline content and pH for an aqueous carrier for administration (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4).

Liquid formulations containing the coated IONRs for parenteral administration may include one or more suspending agents, such as cellulose derivatives, sodium alginate, polyvinylpyrrolidone, gum tragacanth, or lecithin. The liquid formulations may also include one or more preservatives, such as ethyl or n-propyl p-hydroxybenzoate.

Optionally, the liquid formulation containing the coated IONRs contains one or more solvents that are low toxicity organic (i.e., nonaqueous) class 3 residual solvents, such as ethanol, acetone, ethyl acetate, tetrahydofuran, ethyl ether, and propanol, and a combination thereof. Any such solvents included in the liquid formulation should not detrimentally react with the one or more active agents present in the coated IONRs in the liquid fonnulation. Solvents such as freon, alcohol, glycol, polyglycol, or fatty acid, can also be included in the liquid formulation containing the coated IONRs as desired to increase the volatility of the solution or suspension.

Liquid formulations containing the coated IONRs for parenteral administration may also contain minor amounts of polymers, surfactants, or other pharmaceutically acceptable excipients known to those in the art. In this context, “minor amounts” means an amount that is sufficiently small to avoid adversely affecting uptake of the coated IONRs by the targeted cells, such as pituitary gonadotrophs.

The preparation of parenteral formulations containing the coated IONRs is typically under sterile conditions, for example, by lyophilisation, which can be accomplished using standard pharmaceutical techniques known to those skilled in the art.

Formulations for parenteral administration containing the coated IONRs may be formulated to provide immediate and/or modified release of the active agent. Modified release formulations include delayed, sustained, pulsed, controlled, targeted and programmed release formulations.

c. Amount of Coated IONRs

Pharmaceutical compositions typically contain an effective amount of the coated IONRs and one or more pharmaceutically acceptable carriers and/or excipients. As used herein, the term “effective amount” means any amount of the coated IONRs that is sufficient to achieve the desired therapeutic, prophylactic, and/or diagnostic effect on a biological sample or in a subject to which it is administered. Depending on the condition to be prevented, treated, or diagnosed, and/or the route of administration, such an effective amount of the coated IONRs can be between 0.01 to 1000 mg per kilogram body weight of the subject per day, between 0.1 and 500 mg, such as between 1 and 250 mg, for example about 5, 10, 20, 50, 100, 150, 200 or 250 mg, per kilogram body weight of the subject per day, which can be administered as a single daily dose, divided over one or more daily doses. The amount(s) of the coated IONRs administered, the route of administration, and the further treatment regimen can be determined by the treating clinician or testing technologies, depending on factors such as the age, gender and general condition of the subject, the nature and severity of the disease/symptoms being prevented, treated, or diagnosed, and/or the samples being tested.

In some forms, the total amount of the coated IONRs in the pharmaceutical composition can be in a range from about 0.001 wt % to about 20 wt %, from about 0.001 wt % to about 15 wt %, from about 0.001 wt % to about 10 wt %, from about 0.001 wt % to about 5 wt %, from about 0.001 wt % to about 2 wt %, from about 0.001 wt % to about 0.5 wt %, from about 0.001 wt % to about 0.2 wt %, from about 0.001 wt % to about 0.1 wt %, from about 0.01 wt % to about 20 wt %, from about 0.01 wt % to about 15 wt %, from about 0.01 wt % to about 10 wt %, from about 0.01 wt % to about 5 wt %, from about 0.01 wt % to about 2 wt %, from about 0.01 wt % to about 0.5 wt %, from about 0.01 wt % to about 0.2 wt %, from about 0.01 wt % to about 0.1 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2 wt %, from about 0.1 wt % to about 0.5 wt %, or from about 0.1 wt % to about 0.2 wt %. The term “total concentration of the coated IONRs in the pharmaceutical composition” refers to the sum of the weight of the coated IONRs in the composition relative to the weight of the composition.

In some forms, the pharmaceutical composition is in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages can contain between 1 and 1000 mg, and usually between 5 and 500 mg, of the disclosed coated IONRs, e.g., about 10, 25, 50, 100, 200, 300 or 400 mg per unit dosage.

### C. Kits and Devices

Kits and devices containing the coated IONR(s) are disclosed.

In some forms, the user is provided with a kit. The kit includes a device and the coated IONR(s). The device in the kit contains one or more defined region(s) or well(s), which are configured to contain a sample for in vitro applications, such as biomolecule (e.g., cell) separation or detection. The defined region(s) and well(s) of the device can have any suitable volume. For example, each of the defined region or well of the device independently has a volume of less than 100, 50, 25, 10, 5, 4, 3, 2 or 1 cubic centimeters. Optionally, the defined region or well of the device used in the methods is surrounded, lined, or fabricated using a material resistant to water, e.g., glass or plastic. In some forms, the device of the kit contains a plurality of defined regions or wells, e.g., more than or at least 5, 10, 20, 40, 80, 100, 200, or 400 defined regions or wells within one square foot area of the device, such as at least 80 wells within a microtiter plate.

Typically, in the kit, the coated IONRs are stored separately from the device prior to use, such as contained in a package or container separate from the device. In some forms, the coated IONRs are provided in the kit in the form of a pharmaceutical composition, such as in the form of a suspension in a buffered aqueous solution. Optionally, the kit further includes written instructions for performing a test using the device and coated IONRs.

When in use, the user can load the coated IONRs in one or more of the defined region(s) or well(s) and then mix a sample with the coated IONRs in the defined region(s) or well(s) of the device in the kit. Alternatively, the user can mix the coated IONRs with a sample and then load the coated IONRs-sample mixture in one or more of the defined region(s) or well(s) of the device in the kit.

In some forms, the user is provided with just a device containing one or more defined region(s) or well(s) and the coated IONRs. In these forms, the coated IONRs are contained in the defined region(s) or well(s) of the device. Optionally, the defined region or well or each defined region or well of the device is sealed to prevent leaking of the coated IONR(s) contained therein. The coated IONRs contained in the defined region(s) or well(s) of the device may be in solid form and/or liquid form. The defined region(s) or well(s) of the device can have any suitable volume such as any one of those described above. In some forms, the device is configured to contain a plurality of defined regions or wells, such as any one of the configurations described above.

### II. Methods of Making

Methods of making the coated IONRs that have strong magnetic properties are described. Coating iron oxide particles that have strong magnetic properties is a major limitation in the applications of such materials, because they tend to aggregate and form clusters in the coating process, which results in insufficient coating of these materials and thus poor aqueous stability (see, for example, Dynabeads® and magnetic beads supplied by StemCell Technology described above). To achieve sufficient coating of the iron oxide particles, currently available materials use iron oxide particles with relatively weak magnetic properties, leading to coated materials with low separation efficiency (see, for example, magnetic beads supplied by Miltenyi Biotec). The methods disclosed herein can form a sufficient coating on iron oxide nanorods that have strong magnetic properties, and making it possible to produce coated IONRs having both excellent stability in an aqueous medium (i.e., stay suspended in the aqueous medium for at least 1 hour, at room temperature) and superior magnetic property (i.e., a magnetic moment of at least 10 emu/g, induced using 1 T magnetizing field strength, at room temperature).

Generally, the method of making the coated IONRs includes: (i) mechanically mixing (such as by inverting, shaking, or stirring) a mixture comprising IONRs, a coating precursor, and a solvent for a time period in a range from about 1 hour to about 10 hours, from about 1 hour to about 8 hours, from about 1 hour to about 5 hours, from about 2 hours to about 10 hours, from about 2 hours to about 8 hours, or from about 2 hours to about 5 hours, at a temperature in a range from about 60° C. to about 150° C., from about 80° C. to about 150° C., from about 100° C. to about 150° C., such as about 120° C., to form a product comprising the coated IONRs; and (ii) during step (i), periodically sonicating the mixture for at least 1 min, at least 2 mins, at least 5 mins, at least 10 mins, at least 20 mins, at least 30 mins, or in a range from 1 min to 1 hour, from 1 min to 30 mins, from 1 min to 20 mins, from 1 min to 10 mins, or from 1 min to 5 mins, at room temperature. Step (ii) can be performed at a regular time interval or irregular time interval during step (i) mechanical stirring the mixture. For example, step (ii) is performed regularly every 30 mins, every hour, or every 2 hours during step (i), or step (ii) is performed irregularly with a time interval of 30 mins, 1 hour, and/or 2 hours. The IONRs being coated have a magnetic moment of at least 10 emu/g, at least 20 emu/g, at least 50 emu/g, or at least 80 emu/g, induced using 1 T magnetizing field strength, at room temperature.

The coating precursor in the mixture of step (i) can be any sugar (including monosaccharide and disaccharide) suitable for coating IONRs, such as any one of those described above or a combination thereof. For example, the coating precursor in the mixture is glucose. For example, the coating precursor in the mixture is a combination of glucose and another different sugar.

The solvent in the mixture of step (i) is typically a polar organic solvent capable of dissolving the coating precursor, such as glucose. Examples of polar organic solvent suitable for use in the method include, but are not limited to, tetrahydrofuran, chloroform, DMSO, or DMF, or a combination thereof.

Generally, the concentration of the IONRs in the mixture of step (i) can be from about 0.001 mg Fe/mL to about 0.5 mg Fe/mL. The weight ratio between the iron oxide nanorods and the coating material in the mixture of step (i) can be from 500:1 to 5:1, from 100:1 to 5:1, from 50:1 to 5:1, or from 20:1 to 5:1.

Optionally, the disclosed method of making the coated IONRs further includes: sonicating a stock solution comprising IONRs and the solvent for a period in a range from about 1 min to about 1 hour, from about 1 min to about 50 mins, from about 1 min to about 40 mins, from about 1 min to about 30 mins, from about 1 min to about 20 mins, or from about 1 min to about 10 mins, prior to step (i); dissolving the coating precursor in the solvent to form a coating precursor solution at a suitable temperature, such as a temperature ranging from about 60° C. to about 100° C., such as about 90° C., prior to step (i); adding the stock solution into the coating precursor solution, such as by using a syringe pump at a suitable speed (such as a speed ranging from about 0.1 ml/min to 1 ml/min, such as about 0.35 ml/min), to form the mixture, prior to step (i); heating the mixture to the temperature in step (i) (e.g., the mixture is heated such that the temperature is gradually raised to the temperature in step (i)), prior to step (i); and collecting the coated IONRs in the product subsequent to step (i) and (ii), such as by washing the product using a wash solvent (such as ethanol) and subjecting the washed product to ultracentrifugation, where the washing and ultracentrifugation are repeated one or more times, such as one, two, three, four, or five times.

More specific coating conditions and an exemplary method of making the coated IONRs are described in the Examples below.

### III. Methods of Use

The disclosed coated IONRs have excellent aqueous stability and superior magnetic property, and thereby are suitable for use in a wide range of in vivo and in vitro biomedical applications, such as for use in purifying and detecting biological substances, such as proteins, peptides, DNAs, RNAs, cells, extracellular vesicles, and cytokines, in vivo or in vitro, delivering active agents, and imaging as contrast agents, in a subject in need thereof.

A. Separation and/or Detection

In some forms, the disclosed coated IONRs can be used for separating and/or detecting biological substances, such as cells, by employing IONRs which contain a targeting moiety specific for a moiety on the biological substances of interest, such as cells of interest. Typically, the target biological substances, such as target cells, in the sample contain a moiety to which the targeting moiety of the coated IONRs can bind, such that the coated IONRs bind with the target biological substances, such as the target cells, upon mixing. The method generally includes (i) mixing the coated IONRs (containing a target-specific targeting moiety) with a sample containing target biological substances (such as target cells) and non-target substances to form a sample mixture; (ii) exposing the sample mixture to a magnetic field; and (iii) separating the target biological substances from the non-target substances in the sample. Steps (i) and (ii) can be performed sequentially or concurrently. The sample is typically a biological sample of a mammal, such as whole blood, plasma, serum, saliva, nasal swab, mucus, sputum (processed or unprocessed), bronchial alveolar lavage (BAL), bronchial wash (BW), cerebrospinal fluid (CSF), or urine. Upon exposure to the magnetic field, the target biological substances in the mixture are restrained to the magnetic field, thereby allowing non-target substances to be removed from the sample mixture and separated from the target biological substances.

In some forms, the disclosed coated IONRs can be used for detecting the presence of target biological substance(s) in a sample; the method generally includes (i) mixing the disclosed coated IONRs (containing a target-specific targeting moiety) or a composition containing the coated IONRs (containing a target-specific targeting moiety) disclosed herein with the sample to form a sample mixture, such that the coated IONRs bind to target biological substances (such as target cells) in the sample mixture; (ii) exposing the sample mixture to a magnetic field; and (iii) detecting the presence of the target biological substances (such as target cells) in the sample mixture. Separated target biological substances (such as target cells or other disease-related biomarkers) can be detected by methods known in the art, such as fluorescence microscopy after immunofluorescence staining, HPLC, and/or mass spectroscopy. Steps (i) and (ii) can be performed sequentially or concurrently.

In some forms, the methods of separating and/or detecting the presence of target biological substances (such as target cells) described above are performed using a device containing a defined region or well. Typically, the defined region or well of the device has a volume of less than 100, 50, 25, 10, 5, 4, 3, 2 or 1 cubic centimeters. Optionally, the defined region or well of the device used in the methods is surrounded, lined, or fabricated using a material resistant to water, e.g., glass or plastic. In some forms, the device used in the methods described above can contain a plurality of defined regions or wells, e.g., more than or at least 5, 10, 20, 40, 80, 100, 200, or 400 defined regions or wells within one square foot area of the device, such as at least 80 wells within a microtiter plate. When such a device containing one or more defined region(s) or well(s) is used in the methods of separating and/or detecting the presence of target biological substances (such as target cells), the disclosed coated IONRs may be pre-loaded in the defined region or well of the device, or mixed with a sample and then loaded in the defined region or well of the device. When the disclosed coated IONRs are pre-loaded in the defined region or well of the device, a sample is added into the defined region or well and mixed with the coated IONRs and then exposed to a magnetic field capable of restricting the movement of target biological substances (such as target cells) that are bound to the coated IONRs contained in the defined region or well of the device for subsequent separation and/or detection. When the disclosed coated IONRs are supplied separately from the device, a sample is mixed with the coated IONRs and then the sample mixture is loaded into the defined region or well, followed by exposure to a magnetic field for separation and/or detection.

### B. Imaging

In some forms, the disclosed coated IONRs can be used as contrast agents for imaging; the method generally includes (i) administering a pharmaceutical composition containing the coated IONRs to a subject; and (ii) applying electromagnetic radiation to a target region of the subject. In step (ii), one or more sets of image data are obtained and optionally displayed on a device, such as a computer screen. Optionally, after the pharmaceutical composition containing the coated IONRs are administered to the subject, a predetermined time period lapses to allow for uptake of the coated IONRs to a target in the subject, such as cancer cells. The predetermined time for waiting for coated IONRs uptake can be varied depending upon the type of condition/disease and concentration of coated IONRs administered.

In some forms, the target region of the subject being imaged using the disclosed methods can be an organ in the chest and/or abdomen, such as the heart, liver, biliary tract, kidneys, spleen, bowel, pancreas, and adrenal glands; a pelvic organ, such as the bladder, a reproductive organ, such as the breast, uterus, and ovaries in females and the prostate gland in males; blood vessels; brain and brain stem; lymph nodes. In some forms, the disclosed method is to aid in the diagnoses or monitor treatment for conditions, such as tumors of the chest, abdomen or pelvis; diseases of the liver, such as cirrhosis, and abnormalities of the bile ducts and pancreas; inflammatory bowel disease such as Crohn's disease and ulcerative colitis; heart problems, such as congenital heart disease; malformations of the blood vessels and inflammation of the vessels (vasculitis); or a fetus in the womb of a pregnant woman.

The terms “image” and “imaging” refer to a variety of information outputs and associated techniques for gathering useful information from administered coated IONRs and are meant to include all types of external scanning mechanisms for localizing the coated IONRs administered to a subject in need thereof. For example, in one form of basic imaging, spectroscopy is employed as an imaging technique to derive a general determination as to the appearance of coated IONRs and/or the level of coated IONRs in a localized region, which is indicative of the presence of a malignant tumor in the body of a subject to whom the coated IONRs are administered. After such a determination, further imaging can be undertaken (either contemporaneously or after a predetermined time interval) to determine the precise characteristics, size, shape, type, etc. of the area/tissue. This further imaging can employ different and/or more-sensitive imaging devices than those initially employed on the localized areas of coated IONRs. These further imaging devices may or may not be particularly sensitive to the coated IONRs. Such devices include, but are not limited to MRIs, etc. as described herein.

In some forms, the disclosed coated IONRs are used as contrast agents for magnetic resonance imaging (MRI) or magnetic resonance angiography (MRA); the method includes the general steps (i) and (ii) described above and subsequently, a magnetic resonance signal image data set is obtained and optionally displayed on a device, where the magnetic resonance signal image data set is associated with distribution of the coated IONRs in the imaged region of the subject. Optionally, the imaging method disclosed herein further includes administering a second contrast agent to the subject, e.g., comprising Gd³⁺, prior to, concurrently, or subsequent to the administration of the pharmaceutical composition containing the coated IONRs.

### C. Drug Delivery

In some forms, the disclosed coated IONRs can be used for delivering one or more active agents to a subject in need thereof; the method generally includes (i) administering a pharmaceutical formulation containing the coated IONRs to the subject, where the coated IONRs contain one or more active agents embedded in the coating thereof. The administration step can occur one or more times. Typically, the total amount of the one or more active agents that are embedded in the coatings of the IONRs, in the pharmaceutical formulation, is effective to prevent, treat, and/or ameliorate one or more symptoms of a given disease or disorder of the subject in need thereof. The phrase “total amount” with respect to the total amount off the one or more active agents that are embedded in the coatings of the IONRs refers to the sum of the weight of all of the active agent(s) embedded in the coatings of all of the coated IONRs in the pharmaceutical formulation.

Optionally, the method includes administering one or more additional active agents (such as one or more anticancer agents described above), not contained in the coated IONRs, to the subject prior to, concurrently with, or subsequent to administration of the pharmaceutical formulation containing the coated IONRs. These additional active agents not contained in the coated IONRs may be included in the pharmaceutical formulation containing the coated IONRs or in a separate pharmaceutical formulation that does not contain the coated IONRs.

Methods of administration of the pharmaceutical formulations containing the coated IONRs can be oral, i.e., administration to or by way of the mouth, to provide uptake through the GI tract; or parenteral, such as intravenous administration. In systemic circulation, the coated IONRs may accumulate in diseased site.

The pharmaceutical formulations can be administered in a single dose or in multiple doses. Certain factors may influence the dosage required to effectively prevent, treat, or ameliorate the symptoms of a disease or disorder, including, but not limited to, the severity of the disease or disorder, previous preventions, the general health and/or age of the subject, and other diseases present. It will also be appreciated that the effective dosage of the active agent(s) used for prevention or treatment may increase or decrease over the course of particular prevention or treatment. Changes in dosage may result and become apparent from the results of assays.

Preventing a disease or disorder or the symptoms of the disease or disorder includes administering a pharmaceutical formulation containing the coated IONRs to a subject at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, or stabilization or delay of the development or progression of the disease or disorder, or to have a combination of these effects.

1. Diseases or Disorders being Treated

The pharmaceutical formulations described herein can be administered to a subject to prevent or treat any disease or disorder or ameliorate one or more symptoms associated with a disease or disorder.

The subject or patient is an individual who is the target of treatment using the disclosed formulations containing a plurality of the coated IONRs containing one or more active agents embedded in the coatings thereof. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can also include a control subject or a test subject.

Diseases or disorders that can be treated using the drug delivery methods described herein include, but are not limited to, diabetes; autoimmune disorders (e.g. Crohn's disease, chronic arthritis, multiple sclerosis, Sjogren's disease, Lupus erythematosus, psoriasis, Celiac disease, etc); cancer (breast cancer (e.g., metastatic or locally advanced breast cancer), prostate cancer (e.g., hormone refractory prostate cancer), renal cell carcinoma, lung cancer (e.g., small cell lung cancer and non-small cell lung cancer (including adenocarcinoma, squamous cell carcinoma, bronchoalveolar carcinoma and large cell carcinoma)), pancreatic cancer, gastric cancer (e.g., gastroesophageal, upper gastric or lower gastric cancer), colorectal cancer, squamous cell cancer of the head and neck, ovarian cancer (e.g., advanced ovarian cancer, platinum-based agent resistant or relapsed ovarian cancer), lymphoma (e.g., Burkitt's, Hodgkin's or non-Hodgkin's lymphoma), leukemia (e.g., acute myeloid leukemia) and gastrointestinal cancer); pain; fungal infections; bacterial infections; inflammation; anxiety; etc.

The disclosed compositions and methods can be further understood through the following numbered paragraphs.
1. Coated iron oxide nanorods (IONRs) comprising:
   an iron oxide core; and
   a coating,
   wherein the iron oxide core has a magnetic moment of at least 10 emu/g, induced using 1 T magnetizing field strength, at room temperature.
2. The coated IONRs of paragraph 1, wherein the iron oxide core has a length in a range from about 20 nm to about 250 nm and a diameter in a range from about 2 nm to about 50 nm.
3. The coated IONRs of paragraph 1 or 2, wherein the coating comprises an oligosaccharide or a polysaccharide, or a combination thereof.
4. The coated IONRs of any one of paragraphs 1-3, wherein the coating is formed by a monosaccharide or a disaccharide, or a combination thereof.
5. The coated IONRs of any one of paragraphs 1-4, wherein the coating is formed by one or more sugar selected from the group consisting of glucose, fructose, galactose, ribose, xylose, and disaccharides thereof.
6. The coated IONRs of any one of paragraphs 1-5, wherein the coating is formed by glucose.
7. The coated IONRs of any one of paragraphs 1-6, wherein the iron oxide core comprises Fe₃O₄ magnetite or a combination of Fe₃O₄ magnetite and FeO(OH) goethite.
8. The coated IONRs of any one of paragraphs 1-7, having a hydrodynamic length in a range from about 50 nm to about 300 nm, from about 100 nm to about 300 nm, from about 50 nm to about 200 nm, from about 50 nm to about 150 nm, from about 150 nm to about 300 nm, from about 200 nm to about 300 nm, or from about 220 nm to about 300 nm, and a hydrodynamic width in a range from about 20 nm to about 150 nm, from about 20 nm to about 120 nm, from about 20 to about 100 nm, or from about 50 nm to about 150 nm.
9. The coated IONRs of any one of paragraphs 1-8, having a near-neutral surface charge.
10. The coated IONRs of any one of paragraphs 1-9, is dispersed in an aqueous medium for at least 30 mins, at least 1 hour, at least 2 hours, at least 3 hours, or at least 4 hours, at room temperature.
11. The coated IONRs of any one of paragraphs 1-10, having a separation efficiency of at least 80%, at least 85%, at least 90%, or at least 95%, within 1 min of magnet time.
12. The coated IONRs of any one of paragraphs 3-11, wherein one or more of the hydroxyl groups of the coating are converted to an amino, a carboxyl, a thiol, or a maleimide.
13. The coated IONRs of any one of paragraphs 1-12, wherein one or more of the hydroxyl groups of the coating attach to a targeting moiety, optionally wherein the targeting moiety is selected from the group consisting of an antibody, an antibody fragment, an antibody mimetic, an affibody, a nucleic acid, an oligonucleotide, an aptamer, a peptide, a steroid, histidine tag, NTA-Ni, and a tyrosine kinase inhibitor, or a combination thereof.
14. A pharmaceutical composition comprising the coated IONRs of any one of paragraphs 1-13 and a pharmaceutically acceptable carrier and/or excipient.
15. A kit comprising a device and the coated IONRs of any one of paragraphs 1-13, wherein the device comprises one or more defined region(s) or well(s).
16. A device comprising one or more defined region(s) or well(s) and the coated IONRs of any one of paragraphs 1-13, wherein each defined region or well contains the IONRs.
17. A method of producing coated iron oxide nanorods (IONRs), comprising: (i) mechanically mixing (such as by inverting, shaking, or stirring) a mixture comprising IONRs, a coating precursor, and a solvent for a time period in a range from about 1 hour to about 10 hours, from about 1 hour to about 8 hours, from about 1 hour to about 5 hours, from about 2 hours to about 10 hours, from about 2 hours to about 8 hours, or from about 2 hours to about 5 hours, at a temperature in a range from about 60° C. to about 150° C., from about 80° C. to about 150° C., from about 100° C. to about 150° C., such as about 120° C., to form a product comprising the coated IONRs; and
   (ii) during step (i), periodically sonicating the mixture for at least 1 min, at least 2 mins, at least 5 mins, at least 10 mins, at least 20 mins, at least 30 mins, or in a range from 1 min to 1 hour, from 1 min to 30 mins, from 1 min to 20 mins, from 1 min to 10 mins, or from 1 min to 5 mins, at room temperature.
18. The method of paragraph 17, wherein step (ii) is performed regularly every 30 mins, every hour, or every 2 hours during step (i), or wherein step (ii) is performed irregularly with a time interval of 30 mins, 1 hour, and/or 2 hours.
19. The method of paragraph 17 or 18, wherein the IONRs have a magnetic moment of at least 10 emu/g, at least 20 emu/g, at least 50 emu/g, or at least 80 emu/g, induced using 1 T magnetizing field strength, at room temperature.
20. The method of any one of paragraphs 17-19, wherein the coating precursor in the mixture is a sugar, such as glucose, fructose, galactose, ribose, xylose, or a disaccharide thereof.
21. The method of any one of paragraphs 17-20, wherein the coating precursor in the mixture is glucose.
22. The method of any one of paragraphs 17-21, wherein the solvent is a polar organic solvent, optionally wherein the polar organic solvent is tetrahydrofuran, chloroform, DMSO, or DMF, or a combination thereof.
23. The method of any one of paragraphs 17-22, wherein the concentration of IONRs in the mixture is in a range from about 0.001 mg Fe/mL to about 0.5 mg Fe/mL.
24. The method of any one of paragraphs 17-23, wherein the weight ratio between the iron oxide nanorods and the coating material in the mixture is in a range from 500:1 to 5:1, from 100:1 to 5:1, from 50:1 to 5:1, or from 20:1 to 5:1.
25. The method of any one of paragraphs 17-24, further comprising sonicating a stock solution comprising IONRs and the solvent for a period in a range from about 1 min to about 1 hour, from about 1 min to about 50 mins, from about 1 min to about 40 mins, from about 1 min to about 30 mins, from about 1 min to about 20 mins, or from about 1 min to about 10 mins, prior to step (i).
26. The method of any one of paragraphs 17-25, further comprising dissolving the coating precursor in the solvent to form a coating precursor solution at a suitable temperature, such as a temperature ranging from about 60° C. to about 100° C., such as about 90° C., prior to step (i).
27. The method of paragraph 25 or 26, further comprising adding the stock solution into the coating precursor solution, such as by using a syringe pump at a suitable speed (such as a speed ranging from about 0.1 ml/min to 1 ml/min, such as about 0.35 ml/min), to form the mixture, prior to step (i).
28. The method of any one of paragraphs 17-27, further comprising collecting the coated IONRs in the product subsequent to step (i) and (ii), such as by washing the product using a wash solvent (such as ethanol) and subjecting the washed product to ultracentrifugation, optionally wherein the washing and ultracentrifugation are repeated one or more times, such as one, two, three, four, or five times.
29. The method of any one of paragraphs 17-28, further comprising heating the mixture to the temperature in step (i).
30. A method of separating target biological substances comprising:
   (i) mixing the coated IONR(s) of any one of paragraphs 1-13 with a sample comprising the target biological substances and non-target substances to form a sample mixture,
   (ii) exposing the sample mixture to a magnetic field; and
   (iii) separating the target biological substances from the non-target substances in the sample,
   wherein the coated IONRs comprise one or more targeting moieties, and wherein the target biological substances bind to the targeting moieties of the coated IONRs.
31. A method of detecting target biological substances in a sample, comprising:
   (i) mixing the coated IONRs of any one of paragraphs 1-13 with the sample to form a sample mixture;
   (ii) exposing the sample mixture to a magnetic field; and
   (iii) detecting the presence of the target biological substances in the sample mixture,
   wherein the coated IONRs comprise one or more targeting moieties, and wherein the target biological substances bind to the targeting moieties of the coated IONRs.
32. A method of imaging, comprising:
   (i) administering the pharmaceutical composition of paragraph 14 to a subject in need thereof; and
   (ii) applying electromagnetic radiation to a target region of the subject.
33. The method of paragraph 32, wherein in step (ii) one or more sets of image data are obtained and optionally displayed on a visual display.
34. A method of delivering one or more active agent to a subject in need thereof, comprising:
   (i) administering the pharmaceutical composition of paragraph 14 to a subject in need thereof, wherein step (i) occurs one or more times.

### EXAMPLES

### Example 1. Synthesis and Characterization of Exemplary Coated Iron Oxide Nanorods (IONRs)

### Materials and Methods

Two different protocols (Wan J, et al., J. Cryst. Growth. 2005, 276: 571-576; Arijit Mitra, et al., J. Phys. D: Appl. Phys. 2018, 51:085002) were used to synthesize IONRs (also referred to herein as iron oxide core), with modifications. The synthesized IONRs were coated using oligosaccharide.

### IONRs Synthesis Protocol 1

Benzene (8 mL) was laid above 40 ml distilled water in a Teflon liner of 60 ml capacity. Solvents were sonicated for 20 min, followed by purging with argon gas for 20 min. Then 1.0 mmol FeSO₄·7H₂O and 2.0 mmol FeCl₃ were added to be dissolved in the purged distilled water at room temperature. After iron salts dissolved, ethylenediamine (5 mL) was added. The liner was sealed in a stainless-steel autoclave and maintained at 120° C. for 20 h, then cooled to room temperature. The resulting black solid products were filtered off, washed with distilled water and absolute ethanol several times, and dried in oven to yield products in solid form.

### IONRs Synthesis Protocol 2

Synthesis of precursor: 3 g FeCl₃·6H₂O was mixed with varying amounts of PEE (0, 0.1, or 0.2 mL) in 20 mL deionized H₂O under magnetic stirring. The mixture was placed in autoclave and reacted at 120° C. for 4 h.

Synthesis of IONRs: β-FeOOH (100 mg) precursor was dispersed in 10 mL of oleylamine and heated to 120° C. for 15 min, followed by heating to 220° C. for 4-5 h. The reaction was carried out in a strict oxygen-free atmosphere under a constant flow of nitrogen. Oleylamine functions as a solvent, reducing agent, and surface functionalizing agent.

### Oligosaccharide Coating Protocol

Glucose (3 g) in DMF (15 mL) was preheated to 90° C. until completely dissolved. IONRs stock solution (1 mL DMF, 1 mg Fe/mL) was sonicated for 5 min and added into the glucose solution using a syringe pump (0.35 ml/min). The mixture was then gradually (5° C./min) heated to 120° C. and maintained at this temperature for 2.5 h under mechanical stirring with sonication for 1 min at every 30 min. After reaction, the products were precipitated out by the addition of excess ethanol and then collected using ultracentrifuge at 12000 rpm for 20 min. The resultant oligosaccharide IONRs were then washed with ethanol and collected using ultracentrifuge at 12000 rpm for 20 min. The wash-collection cycle was repeated 5 times. The oligosaccharide coated IONRs were then dispersed in DI water.

### Results

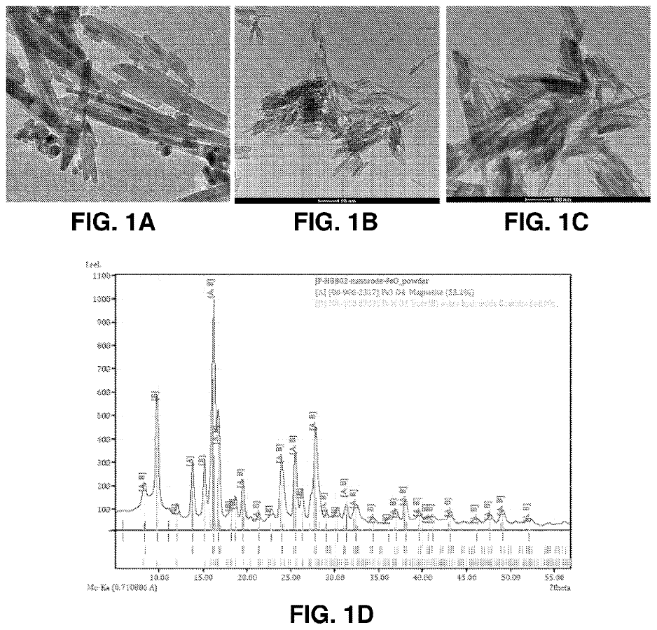

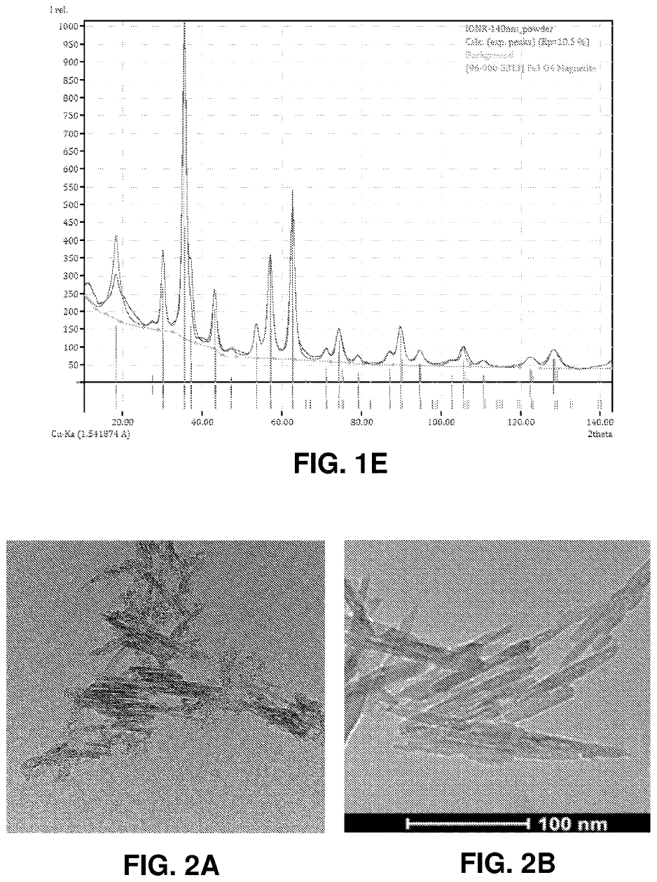

The IONRs were characterized using TEM. The TEM image (scale bar 100 nm) showed that the IONRs synthesized following protocols 1 and 2 have an average length of about 200 nm (protocol 1; FIG. 1A), about 50 nm (protocol 2: FIG. 1B), and about 140 nm (protocol 2; FIG. 1C), respectively. The IONRs synthesized following protocols 1 and 2 were also characterized using XRD, the result of which showed that the IONRs have a mixed composition of Fe₃O₄ magnetite (53.1%) and iron (III) oxide hydroxide goethite (46.9%) (FIG. 1D), or a composition of Fe₃O₄ magnetite (>95%) (FIG. 1E).

The oligosaccharide/polysaccharide coated INORs (produced following protocol 2) were characterized by TEM, dynamic light scattering (DLS). The TEM image and DLS data showed that the oligosaccharide/polysaccharide coated IONRs (which have a core length of about 140 nm) have an aspect ratio (length/width) of about 5 to 10 (FIGS. 2A-2C). DLS measures the hydrodynamic dimensions of the oligosaccharide/polysaccharide coated IONRs. The IONRs before oligosaccharide/polysaccharide coating could not be stably dispersed in water, making the measurement of their hydrodynamic dimensions impossible. Thus, only hydrodynamic dimensions of oligosaccharide/polysaccharide coated INORs could be measured. As shown in FIG. 2C, the oligosaccharide/polysaccharide coated INORs have a hydrodynamic length of about 140 nm.

Particles self-precipitation is an issue that limits use of magnetic particles in biomedical applications. The amount of magnetic materials dispersed in solution was quantified based on the UV absorbance at their corresponding peaks. Particles were vortexed to ensure full dispersion before testing. Absorbance of the fully dispersed solution was set as 100% for data normalization. IONRs synthesized using protocol 2 cannot disperse stably in water. After coating with oligosaccharide, the coated IONRs can stay dispersed in water significantly longer than un-coated ones. Further, as shown in FIG. 2D, the oligosaccharide/polysaccharide coated IONRs were dispersed in water and were stable for a period that is significantly longer than commercially available Dynabeads (Lot #: 00747022) and StemCell Technologies' magnetic beads (Lot #: 1000096771), and similar to Miltenyi's beads (Lot #: 5221009844).

Further, the efficiency of oligosaccharide/polysaccharide coated IONRs pulled out by a magnet from solution was compared with commercially available magnetic beads (i.e., magnetic beads available from Militenyi Biotec (Lot #: 5221009844), Dynabead® (Lot #: 00747022), and magnetic beads available from StemCell Technology (Lot #: 1000096771)). The amount of magnet material dispersed in solution was quantified based on the UV absorbance at their corresponding peaks. Results were normalized with the content before exposure to a magnet set as 100%. As shown in FIG. 2E, the oligosaccharide/polysaccharide coated IONRs (about 140 nm in hydrodynamic length) had a similar efficiency of magnet separation compared to Dynabeads® (4.5 μm in diameter, spherical) and magnetic beads from StemCell Technology (160 nm in diameter, spherical), and a significantly higher efficiency than Miltenyi's beads (50 nm in diameter, spherical). Further, as shown in FIG. 2F, the oligosaccharide/polysaccharide coated IONRs having a hydrodynamic length of about 140 nm showed a magnetic moment (measured using Squid Measurement) of more than 60 emu/g, induced using 1 T magnetizing field strength, at room temperature.

Although Dynabeads® and StemCell Technology's magnetic beads showed a similar efficiency of magnet separation compared to the oligosaccharide/polysaccharide coated IONRs, they showed a much shorter stable dispersion period than oligosaccharide/polysaccharide coated IONRs (FIGS. 2D-2E). Thus, the oligosaccharide/polysaccharide coated IONRs outperform the commercially available magnetic materials (i.e., magnetic beads supplied by Militenyi Biotec, Dynabead®, and magnetic beads supplied by StemCell Technology), since they have both a long period of stable dispersion in water and high separation efficiency by magnet.

1. Coated iron oxide nanorods (IONRs) comprising:
an iron oxide core; and
a coating,
wherein the iron oxide core has a magnetic moment of at least 10 emu/g, induced using 1 T magnetizing field strength, at room temperature.

2. The coated IONRs of claim 1, wherein:
(i) the iron oxide core has a length in a range from about 20 nm to about 250 nm and a diameter in a range from about 2 nm to about 50 nm; or
(ii) having a hydrodynamic length in a range from about 50 nm to about 300 nm, from about 100 nm to about 300 nm, from about 50 nm to about 200 nm, from about 50 nm to about 150 nm, from about 150 nm to about 300 nm, from about 200 nm to about 300 nm, or from about 220 nm to about 300 nm, and a hydrodynamic width in a range from about 20 nm to about 150 nm, from about 20 nm to about 120 nm, from about 20 to about 100 nm, or from about 50 nm to about 150 nm; or
(iii) having a near-neutral surface charge; or
(iv) a combination thereof.

3. The coated IONRs of claim 1, wherein the coating comprises an oligosaccharide or a polysaccharide, or a combination thereof.

4. The coated IONRs of claim 1, wherein the coating is formed by a monosaccharide or a disaccharide, or a combination thereof.

5. The coated IONRs of claim 1, wherein the coating is formed by one or more sugar selected from the group consisting of glucose, fructose, galactose, ribose, xylose, and disaccharides thereof.

6. The coated IONRs of claim 1, wherein the coating is formed by glucose.

7. The coated IONRs of claim 1, wherein the iron oxide core comprises Fe₃O₄ magnetite or a combination of Fe₃O₄ magnetite and FeO(OH) goethite.

8. (canceled)

9. (canceled)

10. The coated IONRs of claim 1,
(i) are dispersed in an aqueous medium for at least 30 mins, at least 1 hour, at least 2 hours, at least 3 hours, or at least 4 hours, at room temperature; or
(ii) having a separation efficiency of at least 80%, at least 85%, at least 90%, or at least 95%, within 1 min of magnet time, or
(iii) a combination thereof.

11. (canceled)

12. The coated IONRs of claim 1, wherein one or more of the hydroxyl groups of the coating are converted to an amino, a carboxyl, a thiol, or a maleimide.

13. The coated IONRs of claim 1, wherein one or more of the hydroxyl groups of the coating attach to a targeting moiety, optionally wherein the targeting moiety is selected from the group consisting of an antibody, an antibody fragment, an antibody mimetic, an affibody, a nucleic acid, an oligonucleotide, an aptamer, a peptide, a steroid, histidine tag, NTA-Ni, and a tyrosine kinase inhibitor, or a combination thereof.

14. A pharmaceutical composition comprising the coated IONRs of claim 1 and a pharmaceutically acceptable carrier and/or excipient.

15. A kit comprising a device and the coated IONRs of claim 1, wherein the device comprises one or more defined region(s) or well(s).

16. A device comprising one or more defined region(s) or well(s) and the coated IONRs of claim 1, wherein each defined region or well contains the IONRs.

17. A method of producing coated iron oxide nanorods (IONRs), comprising:
(i) mechanically mixing (such as by inverting, shaking, or stirring) a mixture comprising IONRs, a coating precursor, and a solvent for a time period in a range from about 1 hour to about 10 hours, from about 1 hour to about 8 hours, from about 1 hour to about 5 hours, from about 2 hours to about 10 hours, from about 2 hours to about 8 hours, or from about 2 hours to about 5 hours, at a temperature in a range from about 60° C. to about 150° C., from about 80° C. to about 150° C., from about 100° C. to about 150° C., such as about 120° C., to form a product comprising the coated IONRs,
wherein during step (i), periodically sonicating the mixture for at least 1 min, at least 2 mins, at least 5 mins, at least 10 mins, at least 20 mins, at least 30 mins, or in a range from 1 min to 1 hour, from 1 min to 30 mins, from 1 min to 20 mins, from 1 min to 10 mins, or from 1 min to 5 mins, at room temperature, and
optionally wherein the sonication is performed regularly every 30 mins, every hour, or every 2 hours during step (i), or wherein the sonication is performed irregularly with a time interval of 30 mins, 1 hour, and/or 2 hours.

18. (canceled)

19. The method of claim 17, wherein the IONRs have a magnetic moment of at least 10 emu/g, at least 20 emu/g, at least 50 emu/g, or at least 80 emu/g, induced using 1 T magnetizing field strength, at room temperature.

20.-29. (canceled)

30. A method of separating target biological substances comprising:
(i) mixing the coated IONR(s) of claim 1 with a sample comprising the target biological substances and non-target substances to form a sample mixture,
(ii) exposing the sample mixture to a magnetic field; and
(iii) separating the target biological substances from the non-target substances in the sample,
wherein the coated IONRs comprise one or more targeting moieties, and wherein the target biological substances bind to the targeting moieties of the coated IONRs.

31. A method of detecting target biological substances in a sample, comprising:
(i) mixing the coated IONRs of claim 1 with the sample to form a sample mixture;
(ii) exposing the sample mixture to a magnetic field; and
(iii) detecting the presence of the target biological substances in the sample mixture,
wherein the coated IONRs comprise one or more targeting moieties, and wherein the target biological substances bind to the targeting moieties of the coated IONRs.

32. A method of imaging, comprising:
(i) administering the pharmaceutical composition of claim 14 to a subject in need thereof; and
(ii) applying electromagnetic radiation to a target region of the subject.

33. The method of claim 32, wherein in step (ii) one or more sets of image data are obtained and optionally displayed on a visual display.

34. A method of delivering one or more active agent to a subject in need thereof, comprising:
(i) administering the pharmaceutical composition of claim 14 to a subject in need thereof, wherein step (i) occurs one or more times.
